(19) United States
(12) Patent Application Publication
(10) Pub. No.: US 2018/0117191 A1
(43) Pub. Date: May 3, 2018

(54) Systems, Devices, and/or Methods for Sterilizing Surfaces

(71) Applicant: Robert Edward Shell, Pocahontas, VA (US)

(72) Inventor: Robert Edward Shell, Pocahontas, VA (US)

(21) Appl. No.: 14/611,066

(22) Filed: Jan. 30, 2015

Related U.S. Application Data
(60) Provisional application No. 61/934,165, filed on Jan. 31, 2014.

(51) Int. Cl. A61L 2/10; A61L 2/00; B65B 55/02

(52) U.S. Cl. CPC A61L 2/10; A61L 2/0047; A61L 2202/21; A61L 2202/23; B65B 55/02

(57) ABSTRACT

Certain exemplary embodiments can sterilize a surface via a system comprising a plurality of ultraviolet emitting light sources. The system can comprise a set of rollers constructed to feed a substantially planar item through the system such that the surface is exposed to ultraviolet light from the plurality of ultraviolet emitting light sources.

### CROSS-REFERENCES TO RELATED APPLICATIONS

This application claims priority to, and incorporates by reference herein in its entirety, pending U.S. Provisional Patent Application Ser. No. 61/934,165 (Attorney Docket No. 2543-02), filed 31 Jan. 2014.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 is a perspective view of an exemplary embodiment of a system 1000;

FIG. 2 is a bottom view of an exemplary embodiment of a system 2000;

FIG. 3 is a side view of an exemplary embodiment of a system 3000;

FIG. 4 is a side view of an exemplary embodiment of a system 4000;

FIG. 5 is a side view of an exemplary embodiment of a system 5000; and

FIG. 6 is a flowchart of an exemplary embodiment of a method 6000.

### DETAILED DESCRIPTION

Certain exemplary embodiments can sterilize a surface via a system comprising a plurality of ultraviolet radiation emitting sources. The system can comprise a set of rollers constructed to feed a substantially planar item through the system such that the surface is exposed to ultraviolet light from the plurality of ultraviolet radiation emitting sources.

Certain exemplary embodiments provide devices, systems, and/or methods allowing sanitizing and/or sterilizing surfaces via ultra-violet (“UV”) radiation substantially without utilizing chemicals or heat. Certain exemplary embodiments comprise a hand-held appliance using light, such as light from any suitable light source to produce the UV radiation and can be powered by a battery pack and/or AC adapter. Certain exemplary embodiments can comprise emitting diodes (“LEDs”) as a light source.

Because many people are very conscious of bacteria and viruses as a danger on many household and other surfaces, a variety of chemicals have been developed to sanitize and/or sterilize such surfaces. However, many people fear or may have allergic reactions to certain chemicals adapted for use in sterilization. Certain exemplary embodiments can be switched on and moved above surfaces to be treated, which can kill and/or inactivate bacteria, fungal spores, parasites, and/or viruses etc. Bacteria and other disease vectors can quickly evolve resistance to chemicals and/or antibiotics used to kill them. However, even after millions of years, bacteria and other disease vectors have yet to evolve resistance to ultraviolet radiation. Studies indicate that certain bacteria and viruses can have exemplary survival times on a dry inanimate surface as follows:
Salmonella—indefinitely;
Rhinovirus—approximately three hours;
Hepatitis C—approximately four days;
Herpes simplex—approximately one week;
SARS (coronavirus)—approximately four weeks; and
MRSA (methicillin-resistant Staphylococcus aureus)—approximately seven months.

Certain exemplary embodiments can be used on dry or wet surfaces. Certain exemplary embodiments are not harmful or destructive to surfaces. Certain exemplary embodiments can, substantially without application of toxic chemicals, kill and/or inactivate germs, bacteria, viruses, and/or amoebic parasites, etc.

The appliance, called SteriLight in certain places herein, uses UV light from LEDs at specific frequencies to kill germs and sterilize surfaces, such as kitchen counters, bathroom counters, toilet seats, tubs, and/or trash cans, etc. Far UV radiation wavelengths shorter than approximately 0.30 microns can be damaging to living organisms. Near UV radiation wavelengths longer than approximately 0.70 microns can activate harmful chemical reactions within microbes, killing them. The SteriLight can be in close proximity to a surface being treated without actually contacting the surface being treated. In some embodiments, the SteriLight can be used in contact with the surface being treated.

In the average kitchen alone there are many surfaces for which sanitization might be desired. Travelers can utilize exemplary embodiments to reduce harmful exposures to microbes. Certain exemplary embodiments can be used on porous surfaces such as sheets and pillowcases.

Certain exemplary embodiments can be utilized as:
denture holders adapted to sterilize dentures;
toothbrush holders adapted to sterilize toothbrushes; and/or
systems adapted to sterilize applicator tips of eye drop dispenser bottles and/or other medication applicators between uses (i.e., eye drops, which can be expensive, would no longer need to be discarded if the applicator tip touched an eye or other surfaces, and/or if a bottle is accidentally dropped on the floor, etc.); etc.

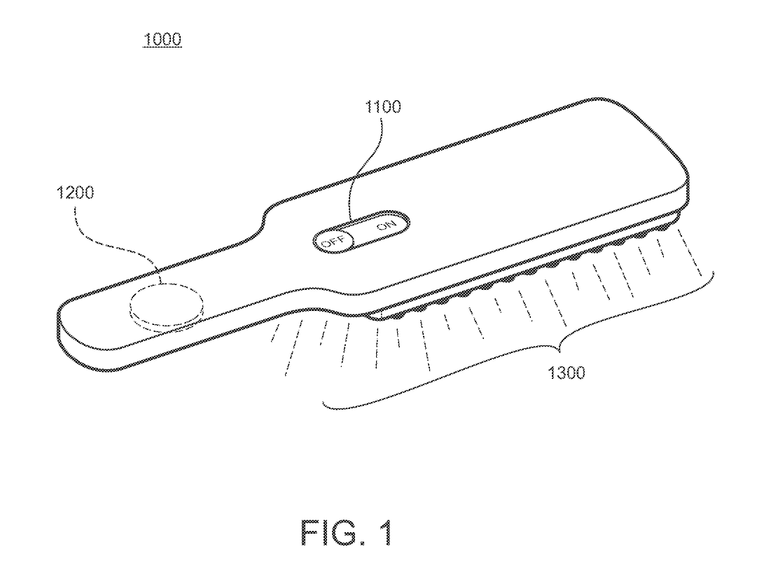

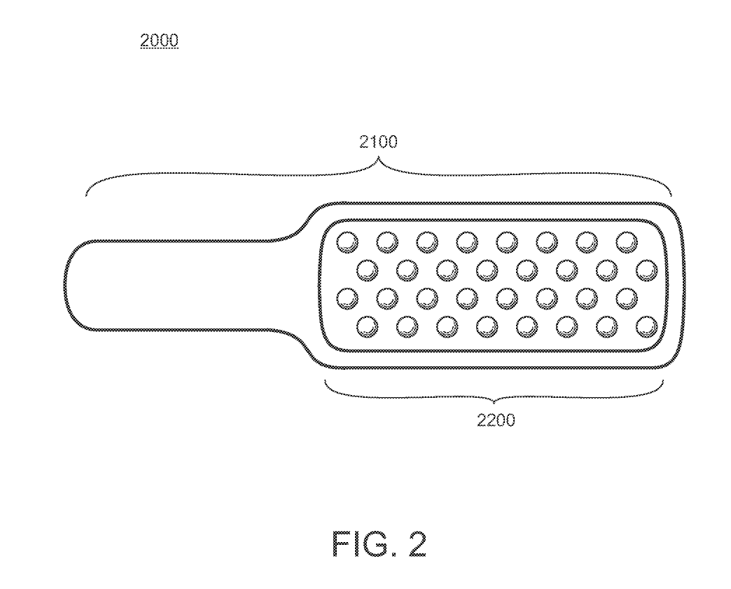

Certain exemplary embodiments provide wand as illustrated in FIG. 1 and FIG. 2, which can be hand-held by a user. The exact number and arrangement of the sources of ultraviolet radiation (e.g., UV-LEDs and/or other UV radiation sources, etc.) varies depending upon a desired application. White light LEDs can be interspersed with UV sources so the user can visually determine an area of coverage.

FIG. 1 is a perspective view of an exemplary embodiment of a system 1000, which can comprise an on/off switch 1100, a power source 1200, and a UV source 1300. In certain exemplary embodiments, power source 1200 can be a battery pack or other compact energy source. In other embodiments, power source 1200 can be electrically coupled to an electrical outlet, such as an outlet providing alternating current electricity and voltages such as approximately, in volts, 120, 240, and/or 480, etc. UV source 1300 can comprise a plurality of lights such as LEDs adapted to emit UV radiation. In certain exemplary embodiments, UV source 1300 can comprise one or more lights adapted to emit light energy in the visible spectrum for humans.

FIG. 2 is a bottom view of an exemplary embodiment of a system 2000, which can comprise a case 2100 and a UV source area 2200. UV source area 2200 can define a plurality of apertures in which one or more sources adapted to emit radiation (e.g., UV radiation emitting sources and/or light energy visible to humans) can be housed. The UV radiation emitting sources can be UV-LEDs and/or other UV radiation sources, etc.

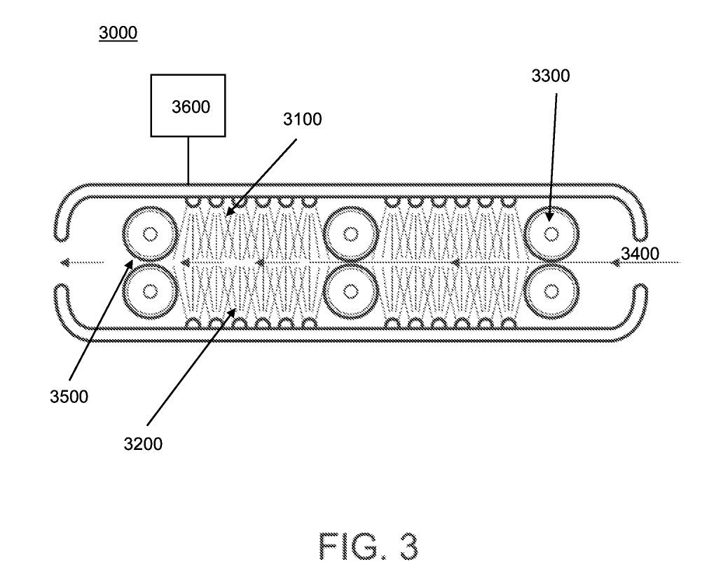

FIG. 3 is a side view of an exemplary embodiment of a system 3000, an exemplary embodiment uses an array of UV sources both above 3100 and below 3200, and rollers 3300, which can be rollers, to feed the items 3400 to be sterilized through the system. The gap 3500 between rollers 3300 can be adjusted, either automatically or manually, to accommodate items of differing thicknesses. Examples of the types of items 3400 that can be sterilized by system 3000 include, but are not limited to, documents, drawings and paintings, currency, cell phones, tablet computers, keyboards, and/or any other substantially planar item that can be fed through system 3000. Since frequently-handled items like currency, restaurant menus, documents of many kinds, which pass through many hands pick up a load of germs, sanitizing them is more and more important as contagious diseases spread in society. Restaurants are now using tablet computers as interactive menus, so some means to sanitize these without chemicals and quickly can be desirable to restaurants. Banks can sanitize currency before passing it on to their customers. Certain exemplary embodiments can sterilize coins with a roller system designed therefor. Certain exemplary embodiments can function substantially without rollers and can utilize other feed through devices and/or methods constructed to move items through such a device and/or system.

Plurality of ultraviolet emitting light sources 3100 and 3200 can be constructed to sterilize a surface of an item 3400. A set of rollers 3400 can be constructed to feed a substantially planar item 3400 through system 3000 such that the surface is exposed to ultraviolet light from plurality of ultraviolet emitting light sources 3100 and 3200. Plurality of ultraviolet emitting light sources 3100 and 3200 can comprise laser-stimulated phosphor lamps. In certain exemplary embodiments, system 3000 can be powered by a battery pack 3600.

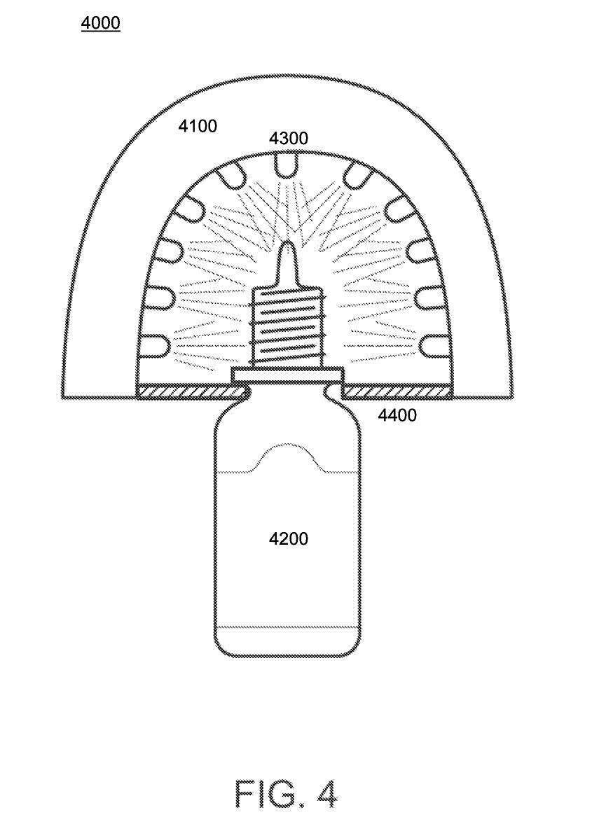

FIG. 4 is a side view of an exemplary embodiment of a system 4000, which can comprise a holder 4100 and a plurality of ultraviolet radiation emitting sources 4300, constructed to hold and sterilize an item 4200. System 4000 can comprise a gasket 4400, which can be made of an elastomeric material to accommodate different sizes of item 4200. Item 4200 can be a medication such as applicator tips of eye drop bottles, and/or other medication applicators, etc. Containers and/or applicators comprising prescription medications, often expensive, can be sterilized. Holder 4100 can be constructed to allow plurality of ultraviolet radiation emitting sources 4300 to sterilize substantially all surfaces of an object. For example, the applicator tip can be sanitized if it is accidentally contaminated. A wipe with a clean cloth or tissue, followed by a quick treatment by inserting the applicator into the sterilizing device can sanitize contagions that might be present.

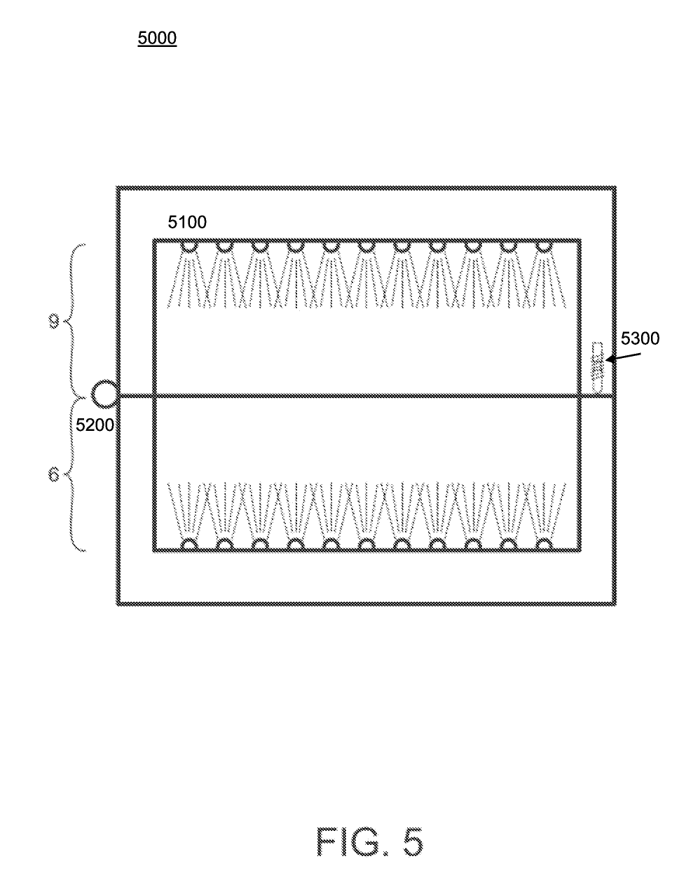

FIG. 5 is a side view of an exemplary embodiment of a system 5000, which can comprise a plurality of ultraviolet radiation emitting sources 5100, a hinge 5200, a switch 5300, an upper portion 9 and a lower portion 6. System 5000 can substantially surround an item being sterilized, such as dentures, contact lenses, artificial eyes, and/or other prosthetics, etc. System 5000 can be opened via a hinge 5200, which can cause a separation between upper portion 9 and a lower portion 6 allowing an object to be sterilized to be placed in system 5000. Switch 5300 can be constructed to turn plurality of ultraviolet radiation emitting sources 5100 on and off. For applications such as dentures, contact lenses, artificial eyes, and/or other prosthetics, etc.; system 5000 can sanitize substantially all surfaces of dentures substantially simultaneously. In certain exemplary embodiments, system 5000 can be used dry. In other embodiments, such as dentures, system 5000 can be filled with water. Some embodiments can be constructed to cause plurality of ultraviolet radiation emitting sources 5100 to turn on when hinge 5200 is in a closed position. Some embodiments can automatically turn plurality of ultraviolet emitting light sources 5100 off after a predetermined time period, such as a user selectable predetermined time for a given item to be sanitized.

Certain exemplary embodiments can utilize laser-stimulated phosphor lamps, recently developed for automotive headlight use, but this technology could be easily adapted to producing UV-C radiation and/or any other wavelengths with germicidal, or sterilizing properties for many types of sterilization.

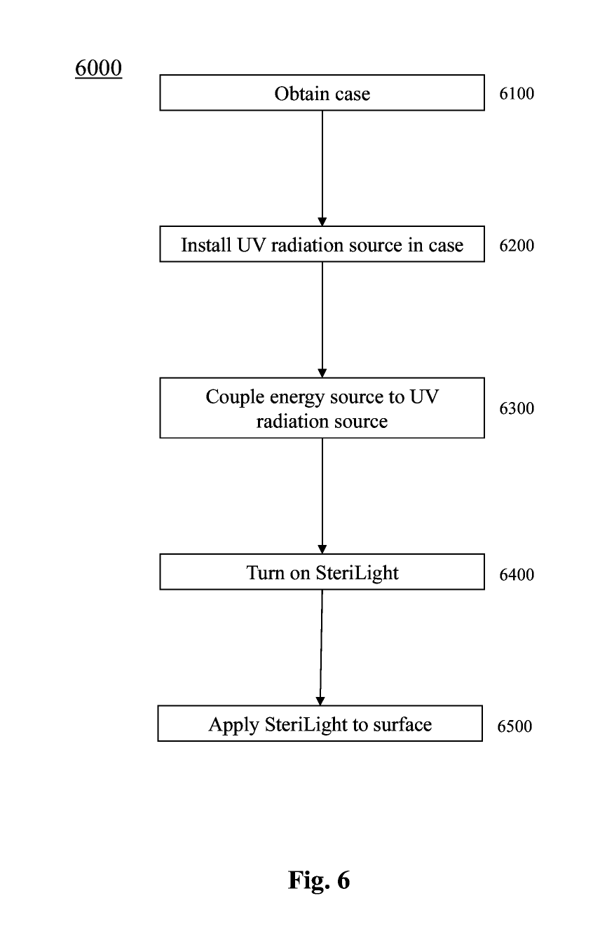

FIG. 6 is a flowchart of an exemplary embodiment of a method 6000. At activity 6100, a case can be obtained. An on/off switch can be present in the case and/or added to the case. An energy source can be present in the case and/or added to the case. At activity 6200, one or more UV radiation sources can be installed in the case. For example, in certain exemplary embodiments, UV radiation emitting LEDs can be installed in the case.

At activity 6300, an energy source can be coupled to the UV radiation source. In certain exemplary embodiments, the energy source can be a battery pack or other source of an electrical direct current. In certain exemplary embodiments, the energy source can be an electrical outlet or other source of an electrical alternating current. At activity 6400, the SteriLight can be turned on. When turned on the SteriLight can emit UV radiation and/or light visible to a human. At activity 6500, the SteriLight can be applied to a surface. The surface can be sanitized and/or sterilized via the application of UV radiation.

Certain exemplary embodiments can sterilize a surface via a system comprising a plurality of ultraviolet emitting light sources. The system can comprise a set of rollers constructed to feed a substantially planar item through the system such that the surface is exposed to ultraviolet light from the plurality of ultraviolet emitting light sources.

Ultraviolet radiation in the approximately 200 to 300 nanometer wavelength range is often called UV-C radiation and/or any other wavelengths with germicidal, or sterilizing properties, and has germicidal capabilities to kill bacteria, viruses, and/or mold spores, etc. UV-C radiation can be used to sterilize water.

The devices, systems, and methods described herein can take one of several forms that use UV-C radiation and/or any other wavelengths with germicidal, or sterilizing properties produced by a UV radiation source such as UV-LEDs (UVEDs), by laser-stimulated phosphor lamps (LSPs), or other UV sources yet to be devised, to sterilize both sides of an object simultaneously, or to sterilize one side at a time when that is more practical.

### Definitions

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.
a—at least one.
activity—an action, act, step, and/or process or portion thereof
adapted to—made suitable or fit for a specific use or situation.
and/or—either in conjunction with or in alternative to.
apparatus—an appliance or device for a particular purpose
associate—to join, connect together, and/or relate.
battery—one or more electrochemical cells constructed to convert stored chemical energy into electrical energy.
battery pack—a set of a count of batteries or individual battery cells configured in a series, parallel, or a mixture of both to deliver a desired voltage, capacity, or power density.
can—is capable of, in at least some embodiments.
cell phone—a telecommunications device that permits a user to conduct a conversation with another over a radio link while moving around a wide geographic area.
comprising—including but not limited to.
currency—money in the form of circulating banknotes.
define—to establish the outline, form, or structure of
denture—a removable prosthetic device constructed to replace missing teeth; when worn by a user, dentures are supported by the surrounding soft and hard tissues of an oral cavity of the user.
device—a machine, manufacture, and/or collection thereof.
document—a written representation of thoughts.
expose—to lay something open such that it is substantially unshielded.
holder—a device and/or system constructed to retain something in a particular location and/or orientation.
keyboard—a hardware device consisting of a number of mechanical buttons (keys) which the user presses to input characters to a computer.
laser-stimulated phosphor lamp—a device and/or system constructed to emit ultraviolet radiation via a luminescent substance that is excited by nearly parallel, nearly monochromatic, and coherent beam of light by exciting atoms to a higher energy level and causing them to radiate their energy in phase.
   may—is allowed and/or permitted to, in at least some embodiments.
medicine—a substance used in treating disease or illness.
method—a process, procedure, and/or collection of related activities for accomplishing something.
object—anything that is visible or tangible and is relatively stable in form.
planar—an object having at least one substantially flat surface.
plurality—the state of being plural and/or more than one.
predetermined—established in advance.
provide—to furnish, supply, give, and/or make available.
receive—to get as a signal, take, acquire, and/or obtain.
repeatedly—again and again; repetitively.
restaurant menu—a list of food items available for sale by a food service establishment.
roller—a cylinder, wheel, caster, or the like, that rotates and upon which something is conveyed along via the rotation.
sterilize—to destroy microorganisms in or on.
surface—an outer face of an object.
substantially—to a great extent or degree.
system—a collection of mechanisms, devices, machines, articles of manufacture, processes, data, and/or instructions, the collection designed to perform one or more specific functions.
tablet computer—a substantially planar programmable electronic device designed to accept data, perform prescribed mathematical and logical operations, and display the results of these operations.
ultraviolet emitting light sources—devices and/or systems configured to act as a source of radiation having wavelengths shorter than approximately 4000 angstrom units.
via—by way of and/or utilizing.

### Note

Still other substantially and specifically practical and useful embodiments will become readily apparent to those skilled in this art from reading the above-recited and/or herein-included detailed description and/or drawings of certain exemplary embodiments. It should be understood that numerous variations, modifications, and additional embodiments are possible, and accordingly, all such variations, modifications, and embodiments are to be regarded as being within the scope of this application.

Thus, regardless of the content of any portion (e.g., title, field, background, summary, description, abstract, drawing figure, etc.) of this application, unless clearly specified to the contrary, such as via explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
no characteristic, function, activity, or element is “essential”;
any elements can be integrated, segregated, and/or duplicated;
any activity can be repeated, any activity can be performed by multiple entities, and/or any activity can be performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any claim element is followed by a drawing element number, that drawing element number is exemplary and non-limiting on claim scope. No claim of this application is intended to invoke paragraph six of 35 USC 112 unless the precise phrase “means for” is followed by a gerund.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

What is claimed is:

1. A system comprising:
a plurality of ultraviolet emitting light sources constructed to sterilize a surface; and
a set of rollers constructed to feed a substantially planar item through said system such that said surface is exposed to ultraviolet light from said plurality of ultraviolet emitting light sources.

2. The system of claim 1, wherein:
the plurality of ultraviolet emitting light sources comprises a laser-stimulated phosphor lamp.

3. The system of claim 1, wherein:
the system is powered by a battery pack.

4. The system of claim 1, wherein:
the substantially planar object is a document.

5. The system of claim 1, wherein:
the substantially planar object is currency.

6. The system of claim 1, wherein:
the substantially planar object is a cell phone.

7. The system of claim 1, wherein:
the substantially planar object is a restaurant menu.

8. The system of claim 1, wherein:
the substantially planar object is a tablet computer.

9. The system of claim 1, wherein:
the substantially planar object is a keyboard.

10. A system comprising:
a plurality of ultraviolet emitting light sources adapted to sterilize a surface; and
a holder constructed to allow said plurality of ultraviolet emitting light sources to sterilize substantially all surfaces of an object.

11. The system of claim 10, wherein:
the object is a denture.

12. The system of claim 10, wherein:
the object is a container constructed to hold medicine.

13. The system of claim 10, wherein:
the object is a toothbrush.

14. A method comprising a plurality of activities, comprising:
sterilizing a surface via a system comprising a plurality of ultraviolet emitting light sources, said system comprising a set of rollers constructed to feed a substantially planar item through said system such that said surface is exposed to ultraviolet light from said plurality of ultraviolet emitting light sources.
